(19) United States
(12) Patent Application Publication
(10) Pub. No.: US 2004/0043445 A1
(43) Pub. Date: Mar. 4, 2004

(54) Oilseed extract products and uses

(76) Inventor: Ralph S. Daniels, Sherman, TX (US)

Correspondence Address: BLODGETT BLODGETT, 43 HIGHLAND STREET, WORCESTER, MA, 016092797

(21) Appl. No.: 10/304,543

(22) Filed: Mar. 11, 2003

Related U.S. Application Data
(63) Continuation of application No. PCT/US01/17121, filed on May 25, 2001.
(60) Provisional application No. 60/207,611, filed on May 26, 2000.
(60) Provisional application No. 60/207,667, filed on May 26, 2000.
(60) Provisional application No. 60/207,597, filed on May 26, 2000.

(51) Int. Cl. A23K 1/00; C12P 1/00

(52) U.S. Cl. 435/41; 426/635

(57) ABSTRACT

The use of fertilizer based on the by-products of refining of agricultural oils or seed extracts, also referred to as “Daniels” or “Daniels Fertilizer” to optimize plant growth and production, optimize the use of microbial inoculation as plant enhancement, the provision of optimum greenness in plants, and the practical use of silicates in oil refining.

### CROSS-REFERENCE TO RELATED APPLICATIONS

This application claims the benefit under 35 U.S.C. § 119(e) of prior U.S. Provisional Patent Applications Nos. 60/207,611; 60/207,667; and 60/207,597, all filed May 26, 2002.

This application is also a continuation of PCT/US01/17121 Designating the United States, filed May 25, 2001. The disclosures of all of these applications are hereby incorporated by reference.

This disclosure also hereby incorporates by reference the content of U.S. Pat. Nos. 4,836,843 and 5,308,372, and the disclosure of the fertilizer formed therein.

### STATEMENT REGARDING FEDERALLY SPONSORED RESEARCH OR DEVELOPMENT

This invention has been created without the sponsorship or funding of any federally sponsored research or development program.

### FIELD OF THE INVENTION

The present invention is directed to fertilizer products and uses based on the byproducts of refining of agricultural oils or seed extracts, also referred to as “Daniels” or “Daniels Fertilizer”.

### BACKGROUND OF THE INVENTION

Prior to the present invention there have been problems optimizing plant growth and production, optimizing the use of microbial inoculation as plant enhancement, obtaining optimum greenness in plants, and using silicates in oil refining. These and other difficulties experienced with the prior art coating systems have been obviated by the present invention.

It is, therefore, a principal object of the present invention to optimize plant growth and production.

A further object of the present invention is to optimize the use of microbial inoculation as plant enhancement.

A further object of the present invention is the provision of optimum greenness in plants.

Another object of the present invention is the practical use of silicates in oil refining. 30 With these and other objects in view, as will be apparent to those skilled in the art, the invention resides in the combination of parts set forth in the specification and covered by the claims appended hereto.

### BRIEF SUMMARY OF THE INVENTION

In its various aspects, the invention involves the use of fertilizer based on the byproducts of refining of agricultural oils or seed extracts, also referred to as “Daniels” or

“Daniels Fertilizer” to optimize plant growth and production, optimize the use of microbial inoculation as plant enhancement, the provision of optimum greenness in plants, and the practical use of silicates in oil refining.

### BRIEF DESCRIPTION OF THE DRAWINGS

FIG. 1 and FIG. 2 show some results of experiments described below in the specification.

### DETAILED DESCRIPTION OF THE INVENTION

Aspects of the invention are described in detail below.

Microbial Inoculant/Biodegradable Carbon Source

The fertilizer of the present invention is a superior carbon source for micro-organism growth/stimulation.

The plant food product, “oilseed extract” and the fortified version of “Daniels 10-4-3” work so well because they provided a superior source of biodegradable carbon available to the indigenous microbes in the growing media. In addition, work has been done with Martin Marietta on their products: 1. Ecomin and 2. SC-27, a biodegradable carbon source on a rock dust substrate and a microbial inoculant. The instant source appears to be superior to Martin Marietta's. Accordingly, a variety of research compared crops (strawberries) grown with Daniels against traditional fertilizers but also against the Martin Marietta products as well as the Daniels with their microbial inoculant. The present invention involves the present fertilizer in combination with microbes alone or with microbes on a substrate. This provides an excellent combination for increasing crop response (yield) as well as for improving other characteristics such as shelf life (senescence) bruising (tomatoes)—all valuable value-added product features.

Both Martin Marietta and Cargill (their Fulcrum line) use molasses as the biodegradable carbon source.

Our “fertilizer” is a combination of fertilizer (mineral and organic nutrient naturally combined with a biodegradable carbon source (seed extracts). This is one of the reasons why the present fertilizer works so well. It is even more effective if it is inoculated or applied with separate applicators, i.e., fertilizer (Daniels/oilseed extract) with microbes.

Oilseed Extract/Daniels 10-4-3 “Greener” Color, Healthier Plants

Plants grown with the plant food of the present invention is that the leaves are 5 universally more green than those plants grown with traditional, water soluble, horticultural grade mineral fertilizers.

The level of iron in our formulation is far less than levels in other plant foods and, it appears, the plant makes better use of the iron (more available) as concluded from the deeper, more vibrant green color versus other feeds.

Professor Paul Nelson at NCSU quantified this work and, pre-publication, concluded the above (deeper/greener color from Daniels Plant Food) is a statistically significant commercial and scientific advantage. Less iron less pollution from unused unnecessary salts which run off and pollute this ground water.

Also the color green is the most appealing to the senses of three basic colors, red, green and blue. A greener plant is a more desirable commercial product. Our plant food produces greener plants with far less iron (the level is below minimum levels for “guaranteed analysis” as determined by the AAPFCO (American Association of Plant Food Control Officials), fertilizer industry's oversight organization and labeling regulatory body.

This feature is a value-added, commercial advantage in, not only the greenhouse plant industry, but also in the nursery markets and the turf (golf courses/greens and the lawns) market.

A greener plant is a healthier plant (actual or perceived) and worth more. The green color improvement is also indicative of a plant which will last longer in the retail ˜nvironment: greater shelf life, more stress (water and heat) tolerant because it is a healthier plant and also more disease resistant. It has been observed that plants grown with Daniels require less pest control chemicals and growth regulators which is a cost saving as well as another environmental benefit.

Silicate Refining/Soapstock

Silicates (sodium and potassium) can be used as the alkali to refine vegetable oils. There have been questions about whether one could tolerate high levels of silicate in our fertilizer.

One aspect of the present invention is to use sodium or potassium silicate to refine vegetable oils. The Na, K— silicate can be used along (singly) or in combination. The soapstock that resulted can be used either as/is as a feed ingredient or fertilizer although it may be better to either evaporate some/all of the water via a spray dryer or wiped film/scraped film evaporator and make a dried, perhaps pelletized, product or the soapstock can first be treated with acid to help dewater the soapstock before use or subsequent drying and pelletizing.

Silicates gel the soapstock and, thereby, stabilize it. This can be used as “granulated” plant/animal food. Also involved is the use of the gelled product for slow release (nutrient) plant food applications such as in potted crops.

The silicate soapstock can be processed via acidulation, also, and the resultant liquid fertilizer (with high silicate content) used as plant or animal foods. Rice crops have high silica requirements for stalk strength. The fertilizer, in any of the above forms (solid, liquid, gel) may result in value-added crop responses. In rice for example, the silica will be assimilated better in the form described above and, together with the other nutrients and organic compounds present from the seed, may act in concert (synergistically) to product a dramatic crop response.

Other Aspects of the Invention

Various aspects of the invention are further described in the following pre-publication manuscript by Professor Paul V. Nelson, Department of Horticultural Science, College of Agricultural and Life Sciences, North Carolina State University, Raleigh, N.C., United States of America, for publication in HortTechnology, a scientific journal of the Amer. Soc. for Hort. Sci. The manuscript is followed by details of a particular experiment by Professor Nelson.

Efficacy of Daniels Soybean-Base Liquid Fertilizer for Greenhouse Crops

ADDITIONAL INDEX WORDS. Dianthus, pansy, petunia, salvia, snapdragon, verbena, vinca, floriculture, ammonium toxicity, substrate pH, electrical conductivity.

### SUMMARY

Three greenhouse experiments were conducted to compare the efficacy of Daniels 1ON:4P₂0₅:3K₂0 fertilize( to various inorganic formulations typical of common greenhouse fertilizers. Crops tested included Petunia xhybsida Hort. ViIm-Andr. ‘Dreams Red’ in the first experiment, Dianthus chinensis L x D barbatus L ‘Floral Lace’, Viola wittrockiana Gams. ‘Crystal Bowl Orange’, Petunia xhybrida Hort. Vilm-Andr. ‘Dreams Red’, Saivia. !aiinacea Benth. ‘Victoria Blue’, Antirrhinum majus L. ‘Bell Mix’, Verbena xhybrida Voss. Quartz Group, Catharanthus roseus (L.) G. Don Cooler Group ‘Grape Cooler’ in the second experiment, and Cyclamen persicum Mill. ‘Laser Rose’ in the third experiment. Foliage of the bedding plants tested in Experiments 1 and 2 was deeper green when fertilized with Daniels fertilizer. No difference in foliar color occurred in cyclarnen plants in Experiment 3. Petunia plants fertilized at a sub-adequate rate were desirably larger when fertilized with Daniels fertilizer compared to inorganic formulations. When fertilized at an adequate rate, dianthus and verbena plants were equivalent in size while pansy, petunia. salvia and vinca plants were more compact when fertilized with Daniels fertilizer compared to a 2N:1P₂Oₛ2K₂O ratio inorganic fertilizer that supplied N in the proportion of 25NH₄:7SN0₃ (25%NH₄). The degree of compaction in Daniels plants was equivalent to that resulting from fertilization with a 2N:1P₂Oₛ2K₂O ratio inorganic fertilizer that supplied N in the proportion o. 75NH₄₅NO₃ (75% NH₄). Compaction was due in large measure to Less leaf expansion Leaf thickness was equal in the compact plants except in petunia where it was greater. Cyclamen plants fertilized with Daniels fertilizer were equivalent in height, width, weight, corn size, and numbers of Leaves and shoots. Flowering occurred earlier in petunia in Experiment 1 and cyclamen in Experiment 3 when fertilized with Daniels fertilizer. Flowering differences did not occur in seven bedding plant taxa in Experiment 2. K deficiency did not occur as a result of fertilizing with Daniels fertilizer in any test plant, although tissue K levels were lower in these plants. Root substrate EC was tower in Daniels compared to inorganic fertilizer treatments in all crops except petunia in Experiment 2 and cyclamen in Experiment 3. Substrate pH was higher for seven taxa in Experiments 2 and 3 in the Daniels compared to the inorganic fertilizer treatments. Nl-1.˜toxicity did not occur in cyclamen in any treatments in Experiment 3 nor in petunia in Experiment I in spite of excessively high rates of N. NH₄ toxicity occurred only in the low (below pH 5.0) substrate pH treatments in Experiment 2. AlL seven taxa developed NH₄ toxicity when fertilized with 75% NH₄. Only pansy developed NH₄ toxicity when fertilized with Daniels fertilizer and the degree of toxicity was Less than that from 75% NH₄.

Greenhouse floral, vegetable, and seedling crops, a small proportion of woody container nursery plants, and most plants in interior consumer and plant-scape settings are fertilized on a continual basis with water soluble fertilizers. A current estimate of consumption of water soluble fertilizers for these purposes is 60,000 short tons (54,500 metric tons) per year in North America (Reddy, SunGrow Horticulture, personal communications). Almost all of this fertilizer is inorganic.

Daniels fertilizer, a recent introduction, is a complete liquid 1ON:4P₂0₅:3K₂0 fertilizer. Although it is not classified as an organic fertilizer, it does contain sufficient biodegradable carbon to give it organic properties. The base for Daniels fertilizer is produced from soybean seed (Daniels, 1996). After roasting and crushing, oils are pressed from the seeds to yield a high value cooking oil product. Following this process, additional lipids are extracted for the animal feed market using NaOH or KOH and neutralization with H₃PO₄. The remaining material is divided into two fractions, solid material containing fiber and protein used for animal feed and soap stock. Soap stock is the aqueous portion of the seeds, It contains minerals and the water soluble compounds including amino acids, organic acids, and sugars. Unless converted-to fertilizer as in Daniels fertilizer, soap stock constitutes a disposal challenge requiring waste treatment to eliminate carbon and lower N content and ultimate discharge of remaining minerals into the environment. For fertilizer production purposes, KOH rather than NaOH is used for lipid extraction from the seeds and additional inorganic nutrients are added to the resulting soap stock to bring it to the guaranteed analysis. Its N composition includes 3.70% NH₄N, 1.90% NO₃—N, 3.65% urea-N and 0.75% organic N. for a total of 81% of N in the reduced forms of NH₄, urea, and organic N. This fertilizer could also be produced from other types of oil seeds such as sunflower, corn, and canola, There is an estimated ???? short tons (???? Metric tons) of oil seed byproduct available for this purpose in the United States per year (REF-2). The benefits to society from adoption of this category of fertilizer are numerable. In addition to relieving the burden on the environment caused by discharge nutrients horn oil seed soapstock, there is the conservation of mineral and energy reserves that would otherwise be consumed in production of fertilizers. Less phosphate and K minerals need be mined from world reserves and less natural gas burned to produce ammonia, the base N stock in water soluble fertilizers.

In addition to conservation, the following reported benefits of biodegradable carbon addition to cropping systems suggest that Daniels fertilizer be investigated for the greenhouselomamental industries. Organic matter additions to soils and soilless substrates can promote soil microbial shifts that suppress pathogenic plant diseases (Hoitink and Boehm, 1999). Soil organic matter can supply nutrients as well as increase cation exchange capacity for holding nutrients for plant use (Chaney, et at., 199?). Soil microbes fostered by compost additions to root substrate can produce organic chetators that hold Fe available to plants in otherwise unavailable situations (Chen, et al., 1998). Microbial assimilation of nutrients during organic matter degradation and subsequent release during mineralization serves to prolong nutrient avauability and minimize loss to the environment (Parnes, 1990).

The grade of Daniels fertilizer and its N content raised three questions that became the basis for the objectives of this study. First, could the single 10-4-3 grade support commercial yield of a wide range of greenhouse crops? Manufacturer's research data and commercial greenhouse acceptance of the product over the past few years suggested that it could. To address this issue eight plant taxa were tested in this study. Second, would the low proportion of K relative to N result in K deficiency? Again, industry use of the product did not indicate existence of this problem. In addition to the range of bedding plant crops, cyclamen was added to the list of test crops in this study because it has been reported that cyclamen has one of the highest K-to N requirements of greenhouse crops (Dole and Wilkins, 1999; VanGall and Wagner, 1996). A 1N:2K₂0 ratio. was recommended by Gerritsen (1998). Third, with 81% of N in one or another reduced forms in Daniels fertilizer, there could he a potential for NH₄ toxicity. In this study, treatments included high levels of Daniels fertilizer to establish adversely high N levels as welt as excessively low substrate pH levels. to encourage NH₄ toxicity since both of these factors have been reported to be associated with increased incidence of NH₄ toxicity (Barker, et al., 1966; Brady, 1990; Forde and Clarkson, 1999).

1. Experiment 1: Petunia

1.1 Materials and Methods

Petunia ‘Dreams Midnight’ plug seedlings were transplanted into 48-cell flats on Apr. 24, 1998. Substrate consisted of equal volumes of sphagnum peat moss and No. 3 grade vermiculite amended with dolomitic limestone, gypsum and MicroMax micronutrient mix (The Scotts Co., Marysville, Okla.) at 8,2, and 1 Ibyd³ (4.7, 1.2, and 0.6 gL⁻¹), respectively. The crop was grown in a glass greenhouse in Raleigh, N.C. at 35° N. latitude that was set at night/cloudy day/clear day temperatures of 62170,75° F. (17/21/24° C.).

A randomized complete block experimental design was used with 12 treatments and three blocks. Each experimental unit consisted of one 48-cell flat of plants. Treatments included five fertilizer sources. See Table 1 for a description of fertilizer treatments and rates of application and Table 2 for formulations of fertilizers. Daniels fertilizer was applied at six N concentrations of 35, 49, 74, 98, 147, and 196 ppm (2.5, 3.5, 5.3, 7, 10.5, and 14 mM). The remaining two fertilizer sources were inorganic formulations that contained a ratio of 2N:1P₂0ₛ:2K₂O and no micronutrients. The N sources were 40:60 and 70:30 ratios of NH₄:N0₃. The 40:60 ratio was selected to be representative of NH₄ content in many commercial greenhouse fertilizers. The 70:30 ratio was selected to represent the urea level in 20N:20P₂0ₛ:20K₂0 fertilizer and offer an unusually high reduced N level similar to that in Daniels fertilizer. These latter two fertilizer sources were applied at three N rates of 49, 98, and 196 ppm (3.5, 7.0, and 14 mM). All fertilizers were applied during each irrigation except on weekends when tap water was applied.

Twenty six days after planting, the average height of plants in each flat was measured and eight plants were selected randomly in each flat for harvest. Plants were cut at the substrate surface, washed in 0.2 N UCI for 30 seconds, rinsed in deionized water, dried at 70° C., weighed, and ground to 1 mm particle size in a stainless steel Wiley mill. Total N was analyzed in this tissue using a Kjeldahl procedure (Fleck, A., 1974). Substrate from these cells was extracted by the saturated media extract procedure (Warncke and Krauskopf, 1983). Extracts were analyzed for pH and EC using a model 695 pH/conductivity/TDS/temperature meter (Extech Instruments, Waltham, Mass). Plants were rated on a scale of 0 (death) to 10 (excellent). Desirable features included deep green foliar color, plant stockiness, and plant size relative to available space in the flat cell. The date when the number of flowers. was equal to 75% of the number of plants present was recorded for each plot. Data were analyzed by ANOVA and means separated by LSD (SAS Inst., Cary, N.C.).

1.2 Results and Discussion

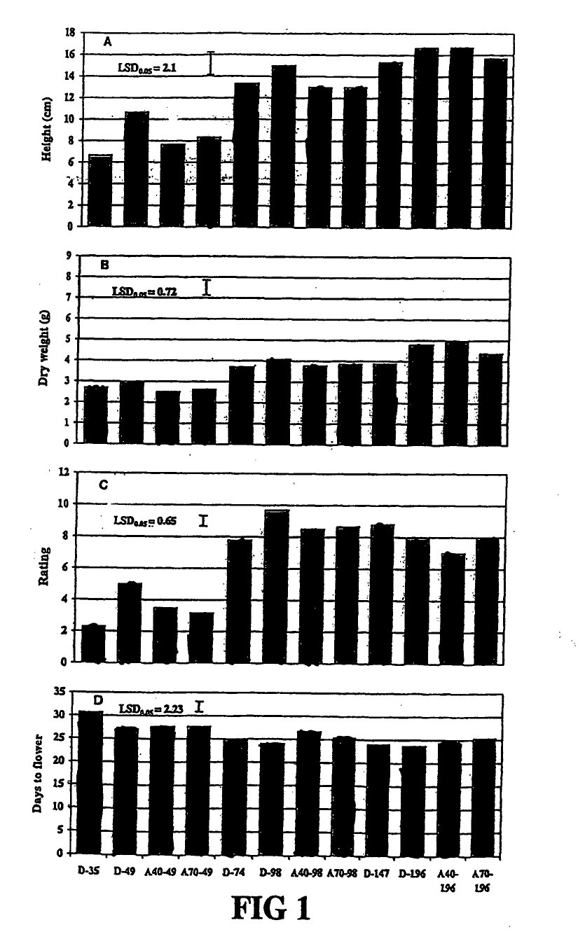

Plants fertilized in treatments 1, 2, 5, 6, 9, and 10 with Daniels fertilizer at N rates from 35 to 196 ppm (mgL⁻¹) developed with increasing N rate, deeper green foliage, taller height (FIG. 1a), and heavier shoot dry weight (FIG. 1b). Height and dry weight were most desirable at the 74 and 98 ppm rates. Plant rating (FIG. 1c) increased up to a N rate of 98 ppm and then declined due to adverse size for the available plant container space. Number of days to flower (FIG. 1d) decreased to a low at 98 ppm and remained at that value at higher N rates. The best rate of application for Daniels fertilizer was 98 ppm. These plants turned out to be the best in the experiment.

Plants in treatments 2-4 that were fertilized with three fertilizers at 49 ppm were lighter green and smaller than desired. Within this series, the best plants were those fertilized with Daniels fertilizer because of deeper green foliage, larger size due to height, earliness of flowering, and highest rating.

The best plants in the experiment were in treatments 5 through 8 where fertilizers were applied at 98 ppm. Within this series, plants fertilized with Daniels fertilizer were taller than plants fertilized with the other two fertilizers. The Daniels fertilized plants flowered in less time than plants fertilized with the 40% NH₄ formulation and were rated higher than the other two treatments.

Plants in the 196 ppm series (treatments 9-12) grew too large for the container resulting in thin stems and a problem with plant toppling. No differences occurred in plant size. Time to flower and ratings were similarly low in all treatments.

Shoot concentrations of N fell into three ranges (Table 3). These ranges were 2.1-2.3%, 2.9-3.2%, and 4.1-4.4% for treatments 1-4, 5-8, and 9-12, respectively. Within each set of treatments the N concentrations did not differ significantly, however they did differ between sets. The two lower ranges are below the survey range reported by Mills and Jones (1996) for petunia. Their survey range does not indicate the minimum critical level. From this experiment it appears that the minimum critical level is around 3% or lower. Growth associated with 4.1% N and higher was too luxuriant for petunia grown within the space confinement of 48-cell bedding plant flats.

No symptoms of K deficiency nor suppression of growth occurred in plants treated with Daniels fertilizer at 98 to 196 ppm compared to plants fertilized with similar concentrations of the two inorganic fertilizer formulations. No symptoms of NH₄ toxicity occurred in any plants in this experiment. This indicates that the margin of safety for NH₄ toxicity from Daniels fertilizer is large since excessive rates of application did not result in toxicity.

While there was a trend for pH to be higher in the Daniels treatment within each N concentration category, the only significant difference occurred in the A70-49 treatment where pH was lower than in the Daniels treatment (Table 3). Within each N concentration category of treatments, Daniels treated substrate was in the lowest EC group.

2. Experiment 2: Bedding Plants

2.1 Materials and Methods

Seven bedding plant taxa obtained as plug seedlings from Ball Seed Co. (West Chicago, Ill.) were transplanted on Jul. 17, 1998 into 48-cell bedding plant flats. The taxa included dianthus ‘Floral Lace’, pansy ‘Crystal Bowl Orange’, petunia ‘Dreams Red’, salvia ‘Victoria Blue’, snapdragon ‘Bell Mix’, verbena ‘Quartz Mix’, and vinca ‘Grape Cooler’. Root substrate consisted of 3 sphagnum peat moss plus 1 horticultural perlite (vv) amended with 3.33 or 10 lbs. finely ground dolomitic limestone in the low pH and high pH series of treatments, 1 lb. Micromnax micronutrient mix (The Scotts Co., Marysville, Ohio), and 1.5 lbs. gypsum per cubic yard of mix (2 or 6, 0.6, and 0.9 gL⁻¹, respectively). Cells were removed from fiats and a block 0112 cells of each taxa was assigned to each experimental unit. The experiment was conducted in a glass greenhouse in Raleigh, N.C. at 35° N. latitude. Temperature was set at nightIc(oudy day/clear day temperatures of 65/75/80° F. (18/24/27° C.).

A randomized complete block experimental plan with three blocks was used. A separate experiment was conducted for each taxa and all were conducted simultaneously. Treatments consisted of a factorial arrangement of two substrate pH levels designated low and high and four fertilizer sources. The low pH level was designed to fall below the acceptable lower limit in order to stimulate NH₄ toxicity. Fertilizer formulations (Table 4) included two inorganic formulations that supplied N:P₂0ₛ:K₂0 in a 2:1:2 ppm ratio similar to 20-10-20 commercial fertilizer. The ratio of ammoniacal to nitrate N in the first was 25:75 and in the second it was 75:25. The first formula was typical of many commercial formulations on the market while the second contained a reduced N (NH₄ content similar to the level of reduced N (NH₄+urea+organic N) in Daniels fertilizer. The higher NH₄ formulation was also selected to enhance NH₄ toxicity. Both formulations were applied at a N concentration of 98 ppm (7 mM). The third and fourth fertilizers consisted of Daniels fertilizer applied at N concentrations of 98 and 196 ppm (7 and 14 mM). The higher concentration was included in the experiment to induce NH₄ toxicity. Fertilizers were applied during each irrigation.

The number of days required to reach 25 percent as many blooms as plants for salvia, 50 percent for dianthus, pansy, snapdragon, verbena, and vinca and 75 percent for petunia were recorded. The experiment for each taxa was terminated when plants reached a marketable state in treatment 1 (high pH—25NH₄:75N0₃). The number of days for this to occur for each taxa was dianthus—41, pansy—18, petunia—26, salvia—18; snapdragon—42, verbena—41, and vinca—40. At market date, plant height was measured from the substrate surface to the top of the plant. Foliar green color was visually assessed in the high pH series of treatments. A scale of 1 to 6 was used with 1 being chlorotic and 6 very deep green. A value of 3 was assigned to fertilizer treatment 1 since that treatment was accepted as the commercial norm. The numbers of flowers and buds were counted. Then the four youngest expanded leaves were removed and their area was measured using a Monochrome Agvision System 286 Image Analyzer (Decagon Devices, Inc., Pullman, Wash.). They were then washed in 0.2N HCl for 30 seconds, rinsed in deionized water, dried for 24 hours at 70° C., and weighed. These leaves from pansy, petunia, salvia, and vinca were ground to 1 mm particle size in a stainless steel Wiley mill and were analyzed for nutrient content. Total N was analyzed using a Kjeldahl procedure (Fleck. A., 1974). Tissue for the remaining nutrients was dry-ashed at 500° C. After addition of 6 N HCI, the ash was heated to dryness at 100° C. to dehydrate it and then dissolved in 0.5 N HCI. P0₄-P was determined by colorimetric analyses (Chapman and Pratt, 1961) using a UV/VIS spectrophotometer (Perkin-Elmer, Norwalk, Conn.). K, Ca, Mg, Fe, Mn, Zn, and Cu were analyzed by atomic absorption spectroscopy. The remainder of the shoot was removed at the substrate surface, dried at 70° C., and weighed. Weights of the four recently expanded leaves and the remainder of the shoot were combined for total shoot dry weight. Substrate was sampled by the saturated media extract procedure (Warncke and Krauskopf 1983). Substrate soluble salt (EC) and pH were determined in each crop as described in the previous experiments. Macronutrients were determined only in pansy, petunia, salvia, and vinca. Colorimetric analysis was performed for NO₃—N (Caltado et al., 1975), NH₄—N (Chaney and Marbach, 1962), P0₄-P concentrations under 10 μgmL⁻¹, Murphy and Riley, 1962; and above 10 μgmL¹, Chapman and Pratt, 1961) on a Model Lambda 3 UV/VIS spectrophotometer (Perkin and Elmer, Norwalk. Conn.). Atomic absorption stectroscopy was used for Ca, Mg, and K analyses.

2.2 Results and Discussion

2.2.1 Foliar Color

Results of visual assessment of the depth of green foliar pigmentation for the four fertilizer treatments in the high pH series are presented in Table 5. Depth of color in 75% NH₄ treated plants, compared to 25% NH₄ plants, was similar in salvia and verbena, deeper in petunia, and lighter in pansy, snapdragon, and vinca. Lighter pigmentation in the latter three taxa was probably due to the early state of NH₄ toxicity. Daniels fertilizer resulted in deeper green color in all six taxa examined. Deeper color has a commercial advantage.

2.2.2 Flowering

Statistical analyses of the number of days to flower, number of buds, and number of flowers showed that there were no effects of the four fertilizer treatments in the high pH series on any of these three variables (data not shown). Performance of Daniels fertilizer was equivalent to the inorganic formulations.

2.2.3 Shoot Growth

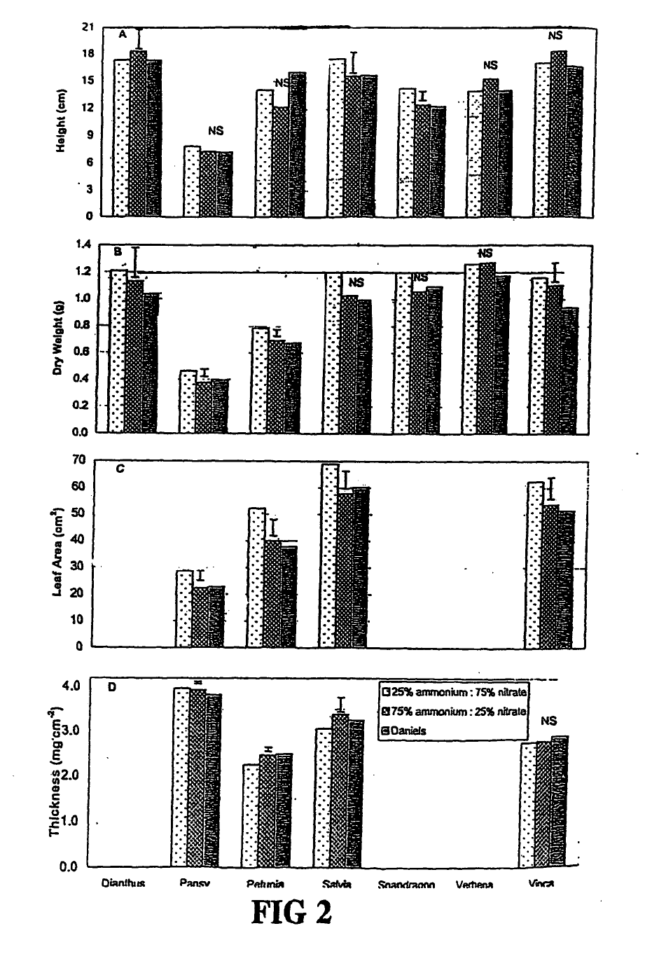

Growth was compared within the three 100 ppm N fertilizer treatments in the high pH series. Neither height (FIG. 2a) nor shoot dry weight (FIG. 2b) of dianthus and verbena plants differed among the Daniels, 25% NH₄, and 75% NH₄ treatments, indicating that Daniels fertilizer performed equivalent to the inorganic formulations for these two taxa. Height and dry weight of snapdragon were similarly lower in the 75% NH₄ and Daniels fertilized plants compared to the 25% NH₄ plants. Plants fertilized with Daniels and 75% NH₄ w&e more compact and fit better into the plant flat space. Within each of the four taxa, pansy, petunia, salvia, and vinca, plant height was equivalent among the three fertilizer treatments. Shoot dry weight was lower in the 75% NH₄ and Daniels treated pansy and petunia plants and in the Daniels treated salvia and vinca plants compared to the 25% NH₄ plants.

The combined area of the four youngest fully expanded leaves was lower in pansy, petunia, salvia, and vinca plants treated with 75% N(−I4 and Daniels than in 25% NH₄ plants (FIG. 2c). All four taxa fit better into the plant flat space when fertilized with 75% NH₄ and Daniels fertilizer due to more compact foliage. Thickness as measured in mass per unit area (FIG. 2d) of the youngest four fully expanded leaves was higher in 75% NH₄ and Daniels compared to 25% NH₄ fertilized petunia plants. Leaf thickness of salvia plants treated with 75% NH₄ was likewise higher than plants fertilized with 25% NH₄. These increases in thickness resulted in exceptionally sturdy and attractive plants. The remaining 75% NH₄ and Daniels treatments did not result in changes in thickness in pansy, salvia, or vinca plants. In general, Daniels fertilized plants were closer in growth and form to plants fertilized with 75% NH₄ than to plants fertilized with 25% NH₄. The former two sets of plants tended to be more compact and better suited to space allotted in bedding plant flats.

General growth assessment of all taxa indicates a strong tendency toward more compact plants when fertilized with a high proportion of NH₄—N. This was also the case for Daniels fertilizer possibly due to the high proportion of N in reduced forms of NH₄, urea and organic N. In bedding plant taxa, compactness is a commercial advantage.

2.2.4 Ammonium Toxicity

No symptoms of NH₄ toxicity developed in any taxa or fertilizer treatment in the high pH series (Table 6). In the low pH series, moderate to heavy NH₄ toxicity occurred in all taxa when fertilized with 75% NH₄. This was anticipated due low substrate pH and high fertilizer proportion of NH₄. Light symptoms developed in pansy and petunia when fertilized with 25% NH₄. By contrast, NH₄ toxicity did not develop in plants fertilized with Daniels at the same 100 ppm N rate in any taxa except pansy. Within the pansy series; fewer symptoms developed in plants treated with Daniels compared to 75% NH₄. Even though Daniels fertilizer contains as much reduced N as the inorganic 75% NH₄ fertilizer, little or no NH₄ toxicity was caused by it. Although it was anticipated that raising Daniels fertilizer concentration from 100 to 200 ppm N would increase NH₄ toxicity, this did not occur. The margin of safety for NH₄ toxicity is very large in Daniels fertilizer.

Symptoms of NH₄ toxicity exhibited some general as well as some specific features across taxa. Dianthus developed interveinal chlorosis on young leaves. Ends of these leaves curled downward in excess of 360°, giving a “pig-tail” appearance. Young leaves on lateral shoots became generally chlorotic. Young leaves of pansy developed chlorosis along the margins that progressed inward into an interveinal pattern. Margins of these leaves rolled upward, sometimes before chiorosis and other times during or after. Young leaves of petunia became chlorotic between veins. Salvia plants developed chlorosis along the margins of young leaves that spread inward to form an interveinal pattern. Moderate distortion occurred on these leaves. Pin point necrotic spots appeared in the chlorotic areas. Snapdragon leaves developed chlorotic blotches 1 mm wide by 2-4 mm long in interveinal regions toward the terminal end of young leaves. As these blotches increased in number and size and spread toward the base of leaves, necrotic spots developed a short distance from the tip of these leaves. The same syndrome later occurred on young leaves of lateral shoots. Young leaves of verbena developed intecveinal chlorosis and margins curled upward. Interveinal chlorosis and upward curling of margins appeared on young leaves of vinca. With time chlorosis spread from the interveinal areas to the entire leaf and faded to a lemon-green color. The terminal end of these leaves were slightly deeper green.

2.2.5 Root Condition

The pattern of root impairment across fertilizer and pH treatments correlated well with the pattern of foliar NH₄ toxicity symptoms reported in Table 6. Within each taxa, roots were healthy and similar in quantity across the fertilizer treatments in the high pH series. Root impairment was restricted to fertilizer treatments in the low pH series. In each case, roots in the low pH series treatments were compared to the general state of roots in the high pH series. Dianthus roots in all low pH fertilizer treatments were similar in white color and quantity to those in the high pH series. In the remaining taxa, roots were reduced in quantity in those treatments where shoot symptoms occurred. This was the case in all taxa when fertilized with 75% NH₄. Pansy and petunia plants fertilized with 25% NH₄ suffered a reduction of roots, but not to the extent of plants fertilized with 75% NH₄. The only Daniels fertilized taxa with reduced root quantity was pansy and the magnitude of this reduction was between that of the 25% NH₄ and 75% NH₄ fertilized plants. The only additional symptom of high NH₄ fertilizer was seen in verbena. Roots on plai˜its fertilized with 75% NH₄ at low pH developed an abnormal tan-brown color and had fewer and shorter root hairs compared to all other treatments. When considering plants in both pH series of fertilizer treatments. Daniels fertilized plants were very resistant to NH₄ toxicity.

2.2.6 Root Substrate pH and EC

Substrate pH levels (Table 7) differed significantly with the largest source of variance due to pH series followed by fertilizer type. The interaction accounted for the least variance and was only significant for petunia, salvia, verbena, and vinca. Substrate pH levels were mainly in the low end of the sate range in the high pH series and below the low end in the tow series. The low series levels were definitely conducive to NH₄ toxicity. As anticipated in both pH series, 75% NH₄ fertilizer lowered pH compared to 25% NH₄ in nearly all comparisons. Substrate pH was equal or higher in the 100 ppm N Daniels treatment compared to 25% NH₄ in all taxa in both pH series. Daniels fertilizer at 200 ppm N resulted in substrate pH equal or higher than that in the 100 ppm N Daniels treatment. Although growth responses from Daniels fertilizer were more typical of a high NH₄ fertilizer, the substrate pH response was characteristic of a tow NH₄ fertilizer. The lower incidence of NH₄ toxicity experienced with Daniels fertilizer compared to the 75% NH₄ fertilizer would be due in only small part to the higher substrate pH in the Daniels treatment. The increase in pH was not sufficient within the low pH series to fully account for the phenomena

Fertilizer, treatments in the high pH series affected substrate salt levels in all taxa except petunia (Table 8). EC levels in the 75% NH₄ treatment were the same as in the 25% NH₄ treatment except in pansy where it was lower. The IX Daniels treatment resulted in lower EC than the 25% NH₄ treatment except in petunia were there was no difference. While EC levels were higher in the 2K Daniels treatment compared to the 1X Daniels treatment they did not differ from those in the 25% NH₄ treatment (100 ppm N). Overall, Daniels fertilizer resulted in lower EC levels than the comparative fertilizers at the same N concentration.

2.2.7 Shoot Nutrient Concentrations

The main effects of fertilizer source and substrate pH on tissue nutrient concentrations were very strong. Their interactive effect was small by comparison. Since variation among fertilizer sources was fairly consistent at both substrate pH levels and differences in tissue nutrient concentrations were great between substrate pH levels, the decision was made to present the interactive data means in Table 9 along with LSD values for the interaction. This was consistent with the goal of the paper, i.e., to relate tissue nutrient concentrations to disorder symptoms within in each treatment where symptoms occurred.

Shoot concentrations of N, P, Mg, and micronutrients were adequate as indicated by published survey ranges (Mills and Jones 1983). Controversial values occurred for K and Ca (Table 9). Pansy tissue K was below the survey range in Daniels 1X treated plants in the low pH series. However, equal symptoms attributed to NH˜ toxicity-appeared in Daniels 2X-low pH plants and more intense symptoms in 75% NH₄-low pH plants, both of which had adequate K according to Mills and Jones (1983). All petunia plants were below the survey range except plants fertilized with 75% NH₄ at high and low pH. Yet, the worst symptoms developed in plants treated with 75%NH₄-low pH that had adequate tissue K. In salvia and vinca, symptoms appeared in plants treated with 75% NH₄-low ph. Plants in both of these treatments had adequate tissue K Symptoms of K deficiency, including marginal necrosis of older leaves or necrotic spots across older leaves, did not occur in this experiment. Based on these data and observations, it can be concluded that K deficiency did not develop, The reported survey ranges specify safe levels but not the minimal critical tissue concentrations of K. These minimum values are unknown.

Tissue Ca concentrations below the survey ranges occurred in pansy, petunia, and vinca plants treated with 75% NH₄, Daniels 1K, and Daniels 2K, all at low pH. Low tissue Ca also occurred in salvia plants fertilized with 75% NH₄ at low pH. While plants in six of these treatments developed symptoms, plants in the remaining four treatments were free of symptoms. This indicates that Ca deficiency was not the sole cause of the symptom syndrome encountered in this experiment.

3 Experiment 3: Cyclamen

3.1 Materials and Methods

Cyclamen ‘Laser Rose’ seedlings produced in 50-cell trays by Wagner Greenhouses, Inc., Minneapolis, Minn. were transplanted into 5.5 inch (14 cm), green, plastic, azalea-type pots, one plant per pot, on Feb. 24, 1999. Root substrate consisted of a mix of 3 sphagnum peat moss to 1 horticultural perlite (v:v) amended with 10 lbs. finely ground dolomitic limestone, 1 lb. Micromax micronutrient mix (The Scotts Co., Marysville, Ohio), and 1.5 lbs. gypsum per cubic yard of mix (6, 0.6, and 0.9 gL⁻¹, respectively). Plants were grown in a double layer polyethylene greenhouse in Raleigh, N.C., latitude 35° N., at temperature settings of 68° F. (20° C.) nights and 73° F. (23° C.) days. The experiment was conducted on 5 ft (1.5 m) wide expanded metal benches with plants spaced 12 in (30 cm) apart in both dimensions. Guard rows were used at both ends of each bench. A randomized complete block design was employed with three treatments and four blocks. Each experimental unit consisted of a single row of five plants across the bench.

Fertilizer treatments were applied with each irrigation (Table 10). Treatment 1 provided a control. It contained the macronutrient equivalent of commercial 20-10-20 (N—P₂0₅-K₂0) fertilizer. As in the commercial product, forty percent of N was ammoniacal with the rest being nitrate. The remaining two treatments consisted of Daniels fertilizer. During the first six weeks, all fertilizers were applied at a N rate of 126 ppm (9 mM). After six weeks, the first two fertilizers were increased to a N rate of 175 ppm (12.5 mM) as is customary in commercial practice. The third treatment remained at 126 ppm to determine if Daniels fertilizer could be used at a lower rate than an inorganic fertilizer.

When the control treatment reached an average of six flowers per plant on May 13 the experiment was terminated. Canopy height was measured as the distance from substrate surface to the upper most part of the leaves. Flower height above the canopy was measured as the difference of canopy height and the distance from the substrate surface to the top of the flowers. Plant diameter was determined by averaging two measurements of plant width taken at right angles to each other. Plant fresh weight included flowers, leaves, crown, and corm. In addition, corm fresh weight was measured and the numbers of shoots, leaves, buds, and flowers were counted.

Substrate was sampled on March 25 two hours after the last application of low concentration fertilizer treatments and on May 13 two hours after the last application of high concentration fertilizer application. Substrate solution was obtained by tipping pots on a diagonal to cause solution to drain from them. Solutions were tested for pH, EC, N0₃-N, NH₄—N, P0₄-P, K, Ca, and Mg using procedures described in the previous experiment. Since treatments 2 and 3 received the same concentration of Daniels fertilizer up to March 25 only treatment 2 was sampled to represent both treatments on the first date.

Recently fully expanded leaves were sampled on March 25 and May 13. Leaves were washed for 30 seconds in 02N Ha, rinsed in deionized water, dried at 70° C. for 24 hours, and ground in a stainless steel Wiley mill to 1 mm particle size. Samples were then analyzed for N, P, K, Ca, Mg, Fe, Mn, Zn, and Cu by procedures described in the previous experiment.

3.2 Results and Discussion

3.2.1 Growth Responses

Growth in the forms of canopy height, flower height above the canopy, plant diameter, plant fresh weight, corm fresh weight, and numbers of shoots, leaves, and buds did not differ across the three treatments (data not shown). For these growth parameters Daniels fertilizer performance was equal to the commercial equivalent fertilizer. The numbers of flowers open on the harvest date of May 13 were significantly different. Plants fertilized with Daniels (Tr. 2) at a concentration of 126 ppm N during the first six weeks and 175 ppm N during the last six weeks had 9.7 flowers compared to 6.6 flowers in the control treatment (Tr. 1) and 7.3 flowers in plants fertilized with a constant rate of Daniels at 126 ppm N throughout the experiment (Tr. 3). The 47% increase in number of flowers in treatment 2 plants could translate into one of two alternative commercial advantages. First, to send a plant to market with more flowers on it after a standard period of production time. Second, to send a plant to market about one week earlier than normal with the standard number of flowers on it. While plants fertilized with the lower rate of Daniels fertilizer in treatment 3 were equivalent in size and number of flowers to control plants they did not have as many flowers as plants fertilized with the higher rate of Daniels fertilizer. It is advisable to use the higher rate of Daniels fertilizer.

3.2.2 Substrate and Tissue Analyses

Substrate pH levels (Table 11) remained nearly constant, in a safe range 5.7 to 5.8, in the Daniels treatments throughout the crop. In contrast, pH level decreased in the control treatment substrate and by the end of the crop reached the lowest safe level of 5.2. Daniels fertilizer provided a buffer against pH decline. Compared to the control, the Daniels treatment receiving the same N concentrations (Tr. 1 vs 2) did not differ significantly in EC and Ca levels, had higher NH₄ and P0₄ levels at the end of the crop, lower NO₃ and K levels at mid and end of the crop, and lower Mg at the end of the crop. Although more N was available in the ammoniacal form in the Daniels treatment, no signs of ammonium toxicity occurred. Tissue (Table 12) in plants fertilized with Daniels fertilizer in treatment 2 compared to control plant tissue was higher in Fe at mid crop and N and Mn at the end of the crop. The elevated levels were in safe ranges. At the middle and end of the crop tissue levels of K and Zn were lower. Mills and Jones (1996) list the minimum adequate levels of K and Zn to be 2.26% and 52 ppm, respectively. Lower K was most likely due to the lower level of K supplied by Daniels fertilizer. Lower Zn could be caused by the higher substrate level of phosphate in the Daniels treatment resulting in precipitation of Zn. Concentrations of K were adequate in alt treatments at the first date and in the control and high rate of Daniels treatments at the second (harvest) date. K in plants fertilized at the low rate of Daniels (Ti. 3) was marginally low at harvest. Although no symptoms of K deficiency appeared in any treatments, the lower rate of Daniels fertilizer would not be advisable. Zn in the control treatment was marginally low on the first date and adequate at harvest. Zn was low on both dates in the Daniels treated plants. The safe tissue level of Zn presented by Mills and Jones (1996) gives no indication of the minimum critical value. For numerous crops a level of 20 ppm of tissue is sufficient (Chapman 1966). On this basis and the lack of Zn deficiency symptoms, Zn was adequate in all treatments.

Literature Cited

Barker, A. V., R. J. Volk, and W. A. Jackson. 1966. Root environment acidity as a regulatory factor in ammonium assimilation by the bean plant. Plant Physiol. 41:1193-1199.

Brady, N. C. 1990. The nature and properties of soils. 10th ed. Macmillian, New York.

Cataldo, D. A., M. Haroon, L. E. Schradei, and V. L. Youngs. 1975. Rapid colorimetric determination of nitrate in plant tissue. Commun. Soil Science and Plant Analysis 6(1):71-80.

Chaney, A. L and E. P. Marbach. 1962. Modified reagents for determination or urea and ammonium. Clinical Chem, 8(2):130-132.

Chaney, [D. E., L E: Drinkwater, arid G. S. Pettygrove. 199?. . . Organic soil amendments and fertilizers. tiC Sustainable Agriculture Research and Educational Program, Univ. of Calif., Div. Agr. and Natural Res. Pub. 21505.

Chapman. H. D. Ed. 1966. Diagnostic criteria for plants and soils. Univ. Bookstore, Univ. of Calif., Riverside, Calif.

Chapman, H. D. and P. F. Pratt. 1961. Methods of analysis for soils, plants, and waters. Univ. of Calif., Div. Agr. Sci. Berkley.

Chen, L. M., W. A. Dick, J. G. Streeter, and H. A. J. Hoitink. 1998. Fe chelates from compost microorganisms improve Fe nutrition of soybean and oat. Plant and Soil 200: 139-147.

Daniels, R. S. 1996. Value-added products from soapstock. Proc. 45th Oilseed Conf., New Orleans, Mar. 10-12, 1996. (Nail. Cottonseed Products Assoc., Inc. arid Southern Region Res. Cen., ARS, USDA).

Dole, J. and H. F. Wilkins. 1999. Floriculture: principles and species. Prentice-Hall, Upper Saddle River, N.J.

Fleck, A. 1974. Micro determination of nitrogen. Critical Rev. Anal. Chem. 4:141.

Gerritsen, H. A. 1998. Cyclamen p. 439-443. In: V. Ball (ed.). The Ball RedBook 16th ed. Ball Pub., Batavia, Ill.

Hoitink, H. A. J. and M. J. Boehm. 1999. Biocontrol within the context of soil microbial communities: a substrate-dependent phenomenon. Annu. Rev. Phytopathol. 37:427-46.

Mills, A. M. and J. B. Jones, Jr. 1996. Plant analysis handbook II. MicroMacro Pub., Inc., Athens, Ga.

Murphy, J. and J. P. Riley. 1962. A modified single solution for the determination of phosphate in natural waters. Anal. Chem. Acta. 27:331-36.

Parnes, R. 1990. Fertilie soil, a growers guide to organic and inorganic fertilizers. agAccess, Davis, Calif.

Van Gall, T. and R. Wagner. 1996. Success with cyclamen—notes on production. Ohio Florists' Assoc. Bul. 805:1,6-8.

Warncke, D. D. and D. M. Krauskopf. 1983. Greenhouse growth media: testing and nutrition guidelines. Mich. State Univ. Coop. Ext. Ser. Bul. E-1736.

TABLE 1
Fertilizer sources and their
concentrations applied in Experiment 1.

| | |
|---|---|
| Treatment | N Concentration |

| No. | Codez | N source | ppm | mM |
|---|---|---|---|---|
| 1 | D -35 | Daniels | 35 | 2.5 |
| 2 | D -49 | Daniels | 49 | 3.5 |
| 3 | A40-49 | 40NH₄:60N0₃ | 49 | 3.5 |
| 4 | A70-49 | 70NH₄:30N0₃ | 49 | 3.5 |
| 5 | D -74 | Daniels | 74 | 5.3 |
| 6 | 0-98 | Daniels | 98 | 7.0 |
| 7 | A40-98 | 40NH₄:60N0₃ | 98 | 7.0 |
| 8 | A70-98 | 70NH₄:30N0₃ | 98 | 7.0 |
| 9 | 0-147 | Daniels | 147 | 10.5 |
| 10 | D-196 | Daniels | 196 | 14.0 |
| 11 | A40-196 | 40NH₄:60N0₃ | 196 | 14.0 |
| 12 | A70-196 | 70NH₄:30N0₃ | 196 | 14.0 |

ZD = Daniels 1 ON:4P₂0₅:3K₂0 fertilizer. A = Inorganic formulation with ratio 2N:1 P205:2K20 where N is derived as specified in ˜N souice column.

Fertilizer treatments. Increased Iron content of plant tissue has also been reported ˜n turl fertilized with Daniels. The substantial differences in plant growth were probably not due to the increased Iron alone. Pansy plants from alt treatments contained sufficient levels of Iron and the shorter internodes and increased axillary breaks observed with pansies receiving Daniels are not consistent with the benefits of elevated [con.

TABLE 3
Tissue nutrient levels of pansies grown at 4
different pnxluction operations. Growers standard
fertility program compared to Daniel's 10-4-3. Nitrogen
rates were approximately the same.

| | | | #2 | | | | | |
|---|---|---|---|---|---|---|---|---|
| | #1 | | Dark | | #3 | | #4 | |
| Location | C. B. Yellow | | Eyed Lemon | | Delta-Yellow | | Majestic mixed | |
| Variety | Total Grow | Daniels | Excel | Daniels | Excel | Dani | Excel | Daniels |
| Fertilizer | 20-0-15 | 10-4-3 | 15-2-20 | 10-4-3 | 15-2-20 | 10-4-3 | 15-2-20 | 10-4-3 |

| | percent | | | | | | | |
|---|---|---|---|---|---|---|---|---|
| N | 5.12 | 4.96 | 5.84 | 5.26 | 4.85 | 4.93 | 5.37 | 5.48 |
| P | 0.54 | 0.66 | 0.71 | 0.64 | 0.68 | 0.76 | 0.96 | 1.10 |
| K | 4.60 | 5.98 | 6.74 | 5.97 | 4.58 | 3.40 | 5.52 | 3.40 |
| Ca | 0.52 | 0.60 | 0.79 | 0.80 | 1.02 | 0.81 | 122 | 1.69 |
| Mg | 0.44 | 0.53 | 0.59 | 0.65 | 0.71 | 0.64 | 0.81 | (IS |
| S | 0.23 | 0.24 | 0.24 | 0.31 | 0.24 | 0.24 | 0.22 | 0.35 |
| | ppm | | | | | | | |
| Fe | 122 | 237 | 128 | iSO | 184 | 217 | 265 | 317 |
| Mn | 80 | 69 | 56 | 31 | 160 | 91 | 429 | 406 |
| 8 | [9 | 21 | 20 | 23 | 19 | 21 | 24 | 31 |
| Cu | tO | 8 | 8 | 7 | 14 | 15 | '2 | 12 |
| Zn | 68 | 74 | 95 | 91 | 62 | 49 | 113 | 156 |
| Mo | 0.81 | 017 | I | 2 | 5 | 0.5 | 0.7 | 0.3 |
| Na | 470 | 480 | 497 | 426 | 574 | 521 | 1992 | 2088 |
| Al | 83 | 44 | 203 | 146 | 564 | 616 | 143 | 156 |

In addition to mineral nutrients, seed endosperm contains many complex organic compounds that arc beneficial to the plant growth. En this authors opinion, the organic fraction (soybean seed extract) of the Daniels fertilizer was a significant contributor to the outstanding quality of the pansy plants fertilized with Daniels. Results provided in Tables 2 and 3 indicate that the pansy plants absorbed similar levels of nutrients when fertilized with a Daniels 10-4-3, Total Grow 15-0-20 or Excel 15-2-20. The dense, compact growth of the pansies fertilized with the Daniels, however, may have been due to a mild growth regulator effect due to some unknown chemical in the soybean seed extract.

Conclusions: Growth and quality of pansies fertilized with Dank.ts 10-4-3 fertilizer were consistently superior to all of the inorganic fertilizers tested. At all trial locations, pansies were larger, more compact, contained more axillary breaks and were of better quality than plants grown with conventional fertility. This fertilizer should be a valuable asset iii pansy fertility management programs.

TABLE 2
Formulations (mM) for the two inorganic
fertilizers sources and the three rates of each applied
in Experiment 1. See Table 1 for a description of
treatments.

| Treat | KNO₃ | NH₄H₂PO₄ | (NH4₂S0₄ | HN₄NO₃ |
|---|---|---|---|---|
| 3 | 1.06 | 0.35 | | 1.05 |
| 4 | 1.06 | 0.35 | 1.05 | — |
| 7 | 2.13 | 0.70 | | 2.10 |
| 8 | 2.13 | 0.70 | 2.10 | — |
| 11 | 4.25 | 1.40 | — | 4.20 |
| 12 | 4.25 | 1.40 | 4.20 | — |

TABLE 3
Whole petunia shoot N concentrations (% of dry
matter), and substrate pH and EC (mS · cm⁻¹) levels 26 days
after transplanting at the end of Experiment 1.

| | Treatment Tissue N | | | Substrate | |
|---|---|---|---|---|---|
| No. | Source N | (ppm) | (% dry wt.) | pH | Substrate EC |
| 1 | Daniels | 35 | 2.2 | 5.2 | 0.74 |
| 2 | Daniels | 49 | 2.3 | 5.4 | 0.70 |
| 3 | 40NH₄:60N0₃ | 49 | 2.2 | 5.2 | 1.18 |
| 4 | 70NH₄:30N0₃ | 49 | 21 | 5.0 | 1.30 |
| 5 | Daniels | 74 | 3.2 | 52 | 0.79 |
| 6 | Daniels | 98 | 3.1 | 5.1 | 0.94 |
| 7 | 40NH₄:60N0₃ | 98 | 2.9 | 5.1 | 0.99 |
| 8 | 70NH₄:30N0₃ | 98 | 3.1 | 4.9 | 1.36 |
| 9 | Daniels | 147 | 4.3 | 5.2 | 0.90 |
| 10 | Daniels | 196 | 4.1 | 5.3 | 1.26 |
| 11 | 40NH₄:60N0₃ | 196 | 4.1 | 5.3 | 1.32 |
| 12 | 70NH₄:30N0₃ | 196 | 4.4 | 5.1 | 2.00 |
| | LSD_{0.05} | | 0.5 | 0.3 | 0.40 |

TABLE 5
Depth of green foliage color in experiment 2.^{z}

| Treatment | Pansy | Petunia | Salvia | Snap. | Verbena | Vinca |
|---|---|---|---|---|---|---|
| 1 | 3 | 3 | 3 | 3 | 3 | 3 |
| 2 | 2 | 4 | 3 | 2.5 | 3 | 2 |
| 3 | 4 | 5 | 3.5 | 4 | 4 | 4 |
| 4 | 5 | 6 | 3.5 | 4 | 4 | 3 |

^{z}Rating 1-6 where 1 very chlorotic, 3 = normal, and 6 = very deep green.
^{y}Lighter green due to early state of NH₄ toxicity.

TABLE 6
Ammonium toxicity rating of taxa in Experiment 2.^{z}

| Treat- | | | | | | | | |
|---|---|---|---|---|---|---|---|---|
| ment | | | | | | | | |
| Fertit- | | | | Petu- | | | Ver- | |
| izer^{y} | pH | Dianthus | Pansy | nia | Salvia | Snap | bena | Vinca |
| 1 | high | 0 | 0 | 0 | 0 | 0 | 0 | 0 |
| 2 | high | 0 | 0 | 0 | 0 | 0 | 0 | 0 |
| 3 | high | 0 | 0 | 0 | 0 | 0 | 0 | 0 |
| 4 | high | 0 | 0 | 0 | 0 | 0 | 0 | 0 |
| 1 | low | 0 | 1 | 1 | 0 | 0 | 0 | 0 |
| 2 | low | 2 | 3 | 2 | 2 | 2 | 3 | 3 |
| 3 | low | 0 | 2 | 0 | 0 | 0 | 0 | 0 |
| 4 | low | 0 | 2 | 0 | 0 | 0 | 0 | |
| 0 | | | | | | | | |

^{z}Rating 0-3 where 0 = no NH₄ toxicity symptoms, 1 = initial symptoms, 3 = extensive symptoms.
See Table 4 for description of fertilizer sources.

TABLE 7
Root substrate pH levels at the end of Experiment 2.

| Treat- | | | | | | | |
|---|---|---|---|---|---|---|---|
| ment | | | | | | | |
| Fertil- | | | | | | | |
| izer | pH | Dianthus | Pansy | Petunia | Salvia | Verbena | Vinca |
| 1 | high | 5.79 | 5.74 | 5.69 | 6.28 | 5.60 | 5.94 |
| 2 | high | 5.40 | 5.43 | 5.43 | 5.06 | 4.71 | 5.03 |
| 3 | high | 5.69 | 5.82 | 5.58 | 6.10 | 5.35 | 5.53 |
| 4 | high | 6.21 | 5.75 | 5.64 | 6.29 | 5.47 | 5.37 |
| 1 | low | 4.17 | 4.42 | 4.41 | 4.69 | 4.25 | 4.69 |
| 2 | low | 3.89 | 4.40 | 428 | 3.95 | 3.88 | 3.66 |
| 3 | (ow | 4.32 | 4.80 | 4.51 | 5.68 | 4.37 | 4.14 |
| 4 | low | 4.79 | 4.90 | 4.77 | 6.69 | 4.90 | 4.85 |
| | LSD_{0.05} | 0.25 | 0.34 | 0.19 | 0.26 | 0.20 | 0.40 |

TABLE 8
Root substrate EC (mS · cm⁻¹) levels at the end of Experiment 2.

| Treat- | | | | | | | |
|---|---|---|---|---|---|---|---|
| ment | | | | | | | |
| Fertil- | | | | | | | |
| izer | pH | Dianthus | Pansy | Petunia | Salvia | Verbena | Vinca |
| 1 | high | 2.12 | 3.52 | 1.61 | 1.29 | 1.41 | 1.34 |
| 2 | high | 2.04 | 2.15 | 1.54 | 1.66 | 1.47 | 1.15 |
| 3 | high | 0.78 | 1.90 | 1.17 | 0.73 | 0.83 | 0.69 |
| 4 | high | 1.85 | 2.19 | 1.23 | 1.32 | 1.23 | 1.39 |
| | LSD_{0.05} | 0.66 | 0.82 | NS | 0.47 | 0.38 | 0.45 |

TABLE 9
Nutrient contents of the four youngest expanded
leaves from treatments in Experiment 2. See Table 4 for
description of fertilizer sources.

| | | | | | | | | | | |
|---|---|---|---|---|---|---|---|---|---|---|
| Treatment | N | P | K | Ca | Mg | ight | Fe | Mn | Zn | Cu |

| | | | |
|---|---|---|---|
| Fertilizer | pH | % dry weight | ppm dry weight |

Pansy

| | | | | | | | | | | |
|---|---|---|---|---|---|---|---|---|---|---|
| 1 | high | 4.45 | 0.58 | 3.78 | 1.83 | 1.36 | 279 | 2517 | 393 | 10.3 |
| 2 | high | 4.18 | 0.55 | 3.91 | 0.81 | 0.87 | 274 | 1677 | 344 | 9.7 |
| 3 | high | 4.95 | 0.65 | 2.88 | 0.83 | 0.90 | 619 | 1116 | 183 | 11.0 |
| 4 | high | 5.70 | 0.80 | 3.40 | 0.64 | 0.75 | 350 | 635 | 171 | 13.3 |
| 1 | low | 4.13 | 0.53 | 3.36 | 1.91 | 1.21 | 255 | 2361 | 407 | 9.0 |
| 2 | low | 3.73 | 0.51 | 3.00 | 0.67 | 0.72 | 54 | 741 | 176 | 7.3 |
| 3 | low | 3.78 | 0.47 | 2.12 | 0.55 | 0.58 | 265 | 482 | 96 | 8.3 |
| 4 | low | 4.99 | 0.67 | 2.63 | 0.50 | 0.64 | 463 | 483 | 115 | 10.0 |
| | LSD_{0.05} | 0.76 | 0.15 | 0.79 | 0.24 | 0.17 | 255 | 649 | 112 | 2.7 |

Petunia

| | | | | | | | | | | |
|---|---|---|---|---|---|---|---|---|---|---|
| 1 | high | 5.70 | 0.43 | 1.99 | 1.97 | 1.63 | 105 | 562 | 100 | 17.7 |
| 2 | high | 5.14 | 0.57 | 3.81 | 0.91 | 0.72 | 109 | 510 | 154 | 21.3 |
| 3 | high | 5.67 | 0.63 | 2.08 | 0.92 | 0.69 | 251 | 420 | 114 | 23.0 |
| 4 | high | 7.29 | 0.94 | 2.46 | 0.79 | 0.51 | 261 | 355 | 104 | 25.7 |

Salvia

| | | | | | | | | | | |
|---|---|---|---|---|---|---|---|---|---|---|
| 1 | low | 5.83 | 0.40 | 2.09 | 1.68 | 125 | 97 | 829 | 161 | 27.3 |
| 2 | low | 5.52 | 0.55 | 4.39 | 0.69 | 0.55 | 161 | 576 | 204 | 33.7 |
| 3 | low | 6.29 | 0.64 | 2.33 | 0.71 | 0.53 | 228 | 473 | 127 | 32.3 |
| 4 | low | 7.32 | 0.91 | 2.97 | 0.63 | 0.44 | 320 | 432 | 116 | 33.0 |
| | LSD_{0.05} | 0.36 | 0.08 | 0.27 | 0.16 | 0.10 | 73 | 98 | 24 | 3.5 |

Vinca

| | | | | | | | | | | |
|---|---|---|---|---|---|---|---|---|---|---|
| 1 | high | 4.91 | 0.39 | 2.21 | 1.64 | 1.68 | 80 | 762 | 110 | 77.3 |
| 2 | high | 4.04 | 0.38 | 2.83 | 1.05 | 1.00 | 151 | 1205 | 220 | 16.3 |
| 3 | high | 4.43 | 0.39 | 1.81 | 1.42 | 1.33 | 552 | 1242 | 146 | 16.3 |
| 4 | high | 6.87 | 0.73 | 2.44 | 1.04 | 0.94 | 692 | 887 | 127 | 22.7 |
| 1 | low | 5.57 | 0.44 | 2.56 | 2.12 | 1.34 | 118 | 1034 | 269 | 21.7 |
| 2 | low | 4.74 | 0.42 | 2.91 | 0.83 | 0.74 | 159 | 1366 | 340 | 25.0 |
| 3 | low | 5.32 | 0.46 | 2.18 | 1.48 | 1.16 | 1015 | 1721 | 234 | 21.0 |
| 4 | low | 6.86 | 0.68 | 2.94 | 0.99 | 0.82 | 1355 | 1160 | 144 | 22.3 |
| | LSD_{0.05} | 0.38 | 0.06 | 0.18 | 0.28 | 0.22 | 287 | 263 | 35 | 2.7 |

Vinca

| | | | | | | | | | |
|---|---|---|---|---|---|---|---|---|---|
| High | 6.09 | 0.50 | 1.81 | 2.22 | 1.19 | 113 | 441 | 119 | 14.0 |
| High | 5.80 | 0.61 | 2.60 | 1.30 | 0.62 | 111 | 552 | 170 | 17.3 |
| High | 5.76 | 0.57 | 1.41 | 124 | 0.62 | 129 | 367 | 102 | 15.7 |
| High | 7.30 | 0.66 | 2.20 | 0.95 | 0.48 | 133 | 361 | 95 | 18.7 |
| Low | 6.21 | 0.51 | 1.73 | 1.98 | 0.98 | 132 | 733 | 137 | 14.7 |
| Low | 5.54 | 0.61 | 2.97 | 0.81 | 0.52 | 76 | 824 | 170 | 17.3 |
| Low | 5.82 | 0.62 | 1.59 | 0.74 | 0.49 | 123 | 602 | 104 | 16.7 |
| Low | 7.39 | 0.70 | 2.24 | 0.66 | 0.37 | 130 | 486 | 76 | 17.0 |
| LSD_{0.05} | 0.34 | 0.04 | 0.34 | 0.11 | 0.08 | '10 | 75 | 18 | 2.3 |

TABLE 10
Nutrient sources and concentrations (mM) used
in three fertilizer treatments in Experiment 3.

| | |
|---|---|
| Nutrient | Concentration (mM) |

| Treatment | source | Days 1-42 | Days 43-78 |
|---|---|---|---|
| I | NH₄H₂PO₄ | 0.6 | 0.83 |
| | KNO₃ | 2.7 | 3.75 |
| | NH₄NO₃ | 2.7 | 3.75 |
| | (NH₄)₂SO₄ | 0.15 | 0.21 |
| | (Total N) | 9.0 | 12.5 |
| 2 | Daniels | | |
| | (Total N) | 9.0 | 12.5 |
| 3 | Daniels | | |
| | (Total N) | 9.0 | 9.0 |

TABLE 12
Nutrient content of recently mature leaves of
cyclamen in Experiment 3.

| | | | | | | | | |
|---|---|---|---|---|---|---|---|---|
| N | P | K | Ca | Mg | Fe | Mn | Zn | Cu |

| | | |
|---|---|---|
| Treat. | % dry wt. | ppm² |

Mid-crop

| | | | | | | | | | |
|---|---|---|---|---|---|---|---|---|---|
| 1 | 3.49 | 0.33 | 3.70 | 0.41 | 0.37 | 49 | 130 | 48 | 7.8 |
| 2 | 3A8 | 0.38 | 2.69 | 0.42 | 0.40 | 85 | 127 | 24 | 7.6 |
| LSD₀₀₅ | NS | NS | 0.18 | NS | NS | 22 | NS | 13 | NS |

End-crop

| | | | | | | | | | |
|---|---|---|---|---|---|---|---|---|---|
| 1 | 3.48 | 0.33 | 3.54 | 0.84 | 0.44 | 99 | 92 | 84 | 8.5 |
| 2 | 3.77 | 0.35 | 2.66 | 1.06 | 0.53 | 146 | 119 | 35 | 7.8 |
| 3 | 3.52 | 0.30 | 221 | 0.88 | 0.51 | 98 | 105 | 35 | 7.4 |
| LSD_{0.05} | 0.07 | NS | 0.37 | NS | NS | NS | 13 | 7 | NS |

^{z}ppm = μg˜g⁻¹

FIG. 1. Height (a), shoot dry weight (b), and plant rating (c) (1=poor to 10=excellent) 26 days after transplanting and days from transplant to the point where the number of flowers equaled 75% of the number of plants (d) for petunia ‘Dreams Midnight’ bedding plants in Experiment 1. Treatment code: D=Daniels 1QN:4P₂0₅:3K₂0 fertilizer, A 40=inorganic 2N:1P₂0₅:2K₂0 fertilizer with N consisting of 40NH₄:60N0₃, A70=inorganic 2N:1 P₂0₅:21K₂0 fertilizer with N consisting of 70NH₄:30N0₃, and the number following the fertilizer source, 35, 49, 74, 98, 147, or 196,=N concentration (ppm, mg:L⁻¹).

FIG. 2. Height (a), shoot dry weight (b), area of the youngest four expanded leaves (c), and thickness of the youngest four expanded leaves expressed as dry weight per unit leaf area measured at marketable date for each of seven bedding plant taxa in Experiment 2.

### Daniels-8-99

### 12-4-00

Subject

Daniels Fertilizer as a nutrient source and an activator of Martin Marietta EcoMin and SC27 on Plasticulture Strawberries.

Purpose

1. Compare the effectiveness of Daniels fertilizer to conventional inorganic fertilizer.

2. Test the benefit of the biodegradable carbon in Daniels fertilizer to EcoMin and SC27.

Procedure

Starting Date—Strawberry plants transplanted into beds on Oct. 13, 1999.

Test Crops—‘Chandler’ strawberry plants from Canada.

Test Location—Central Crops Research Stat ion (NCSU) 13223 US 70 West, Clayton, N.C. 27520-2127. (western zone of the coastal plain)

Bed Description—Six 75 ft. long beds 24 in. wide and 8 in. high were formed with a black polyethylene cover and a P-tape dripper buried 2 in. deep along the center of the bed. Each bed was divided into 5 plots each 10 ft. long and each separated by a 5 ft. fallow space. Twenty plants were arranged in each. plot in a double row with plants 1 ft apart within the row and rows 1 ft apart.

Soil Type—Norfolk sandy loam.

Design—10 treat×3 rep=30 plots (20 plants/plot=600 plants).

Fertilizer

At Planting: Determine the quantities of materials to apply on the basis of 50 sq. ft. per plot, 5×10 ft.

Conventional full rate at planting time N, P₂0₅, K₂0 at 60, 30, and 120 lbs per acre from ammmonium nitrate, triple superphosphate, and potassium sulfate. Broadcast this fertilizer over the top of the bed and rototill it in.

Daniels full rate: at planting time 60 lbs N per acre froni Daniels fertilizer was applied by diluting Daniels fertilizer in 1.5 L of well water, sprinkling it over the top of the preformed bed, and rototilling it in.

In the Spring: We based our applications on 6O ft² of bed (5×12 ft serviced by a 12 ft T-tape). On three dates, March 17, April 6, and 24 we applied fertilizer, as listed below, through the T-tape dripper. This field was known to be low in S and B. We applied Solubor (20% B) once on March 17 at the rate of 0.125 lbs B/acre, (1.2 g Solubor/180 ft² −3 reps of a treatment). We applied Epsom salts (13% Mg) on each of the 3 spring fertilization dates at the rate of 20 lbs S/acre (96.4 g Epsom salt/l8O ft² −3 reps of a treatment).

Fertilizers Applied in the Spring

1. Water (treatment 1)

2. 100% conventional (20 lbs N/acre) : 36.84 g NH₄N₃/6O ft² plot (treatment 3). [110.5 g for 3 plots]

3. 75% conventional (15 lbs N/acre) : 27.63 g NH₄N₃/6O ft² plot (trt. 2, 5, 6, 7, 8). [414.5 g for 15 plots]

4. 100% Daniels: (20 lbs N/acre): 125 g Daniels liquid fertilizer/6O ft² plot (trt. 4, 10). [750 g for 6 plots]

5. 75% Daniels: (15 lbs N/acre): 94 g Daniels liquid fertilizer/6O ft² plot (trt. 9). [282 g for 3 plots]

Calculations

1 acre=43,500 ft²; 60 1W43,500 ft²=0.0013793;

20 lbs N×454 g/lb=9,080 g N/acre;

9,080 gNxO.0013793=12.524 gN/60 ft² plot;

12.524 g N/0.34=36.84 g NH₄N₃/6O ft² plot;

125.24 g N/0.10=125.24 g Daniels fertilizer/6O ft² plot;

Treatments

An unfumigated and unfertilized field was disked and beds were preformed with disks on October 12. Water tape and plastic sheeting were not applied at this time. On October 13 fertilizers, EcoMin and 5C27 were applied to the surface of each assigned plot. SC27 was diluted 10 fold with deionized water and then. the appropriate amount was diluted to L5 L in a watering can and sprinkled over the plot. Finally, each plot was rotolilled to incorporate the materials into the soil. The bedding equipment was drawn over the bed again to reform it and apply water tape and plastic covering.

TABLE 1
Description of treatments*.

| | | | EcoMin | SC-27 |
|---|---|---|---|---|
| TrL | % Fert | Fertilizer | (lbs/A) | (l/A) |
| 1 | 0 | none | 0 | 0 |
| 2 | 75 | conventional | 0 | 0 |
| 3 | 103 | conventional | 0 | 0 |
| 4 | 100 | Daniels | 0 | 0 |
| 5 | 75 | conventional | 250 | 0 |
| 6 | 7 | conventional | 0 | 1 |
| 7 | 75 | conventional | 250 | 1 |
| 8 | 75 | conventional | 250 | 2 |
| 9 | 75 | Daniels | 250 | 1 |
| 10 | 1(X) | Daniels | 250 | 1 |

*Quantities per 50 ft² plot. ammonium nitrate 100% = 91.9 g, 75% = 68.9 g; triple superphosphate 100% = 34.7 g. 75% = 26.0 g potassium sulfate 100% = 125.0 g, 75% =

Randomization

| | | |
|---|---|---|
| Rep 1 | Rep 2 | Rep 3 |

| | | | | | |
|---|---|---|---|---|---|
| 2 | 1 | 3 | 9 | 10 | 7 |
| 6 | 4 | 7 | 1 | 2 | 4 |
| 8 | 5 | 2 | 10 | 8 | 5 |
| 9 | 10 | 5 | 4 | 3 | 9 |
| 3 | 7 | 6 | 8 | 1 | 6 |

Pest Control

March 24: Captan+Topsin-M for botrytis prevention.

April 1: Elevate for fungal prevention

April 6: Elevate for fungal prevention

Date Sought

Total fresh weight of ripe fruit, fresh weight of reject fruit (cull wt), net weight of fruit (total—cull), average weight of a fruit (average of a 25 fruit random sample) on each sample date. Sample 2 times per week on M and El or T and F.

Results

Dec. 16, 1999 Plots were weeded over the past 2 weeks. All plants survived. New growth is at least 5 times that which was planted. Two treatments appear to stand out today. Plants in Tr. 1, without fertilizer, EcoMin or SC-27, are smaller and show a little chiorosis typical of N deficiency. Plants in Tr. 10 that received the 100% rate of Daniels plus EcoMin and SC-27, appear to be deeper green and larger, due to larger leaves and possibly more of them. It is difficult to see differences among the remaining treatments.

Mar. 9, 2000 Rating on a scale of 0 dead to 10 excellent plant size and color.

| | Rep. | | | |
|---|---|---|---|---|
| Tr. | 1 | 2 | 3 | Ave |
| 1 | 7 | 7 | 9 | 7.7 |
| 2 | 8 | 9 | 9 | 8.7 |
| 3 | 7 | 8 | 9 | 8.0 |
| 4 | 10 | 9 | 8 | 9.0 |
| 5 | 8 | 10 | 8 | 8.7 |
| 6 | 9 | 8 | 8 | 8.3 |
| 7 | 8 | 8 | 8 | 8.0 |
| 8 | 7 | 8 | 8 | 7.7 |
| 9 | 7 | 8 | 7 | 7.3 |
| 10 | 10 | 9 | 10 | 9.7 |

April 6: First red fruit.

April 13 to June 2: Fruit was harvested in this period. Throughout this period of time treatment 10 continued to have noticeably larger plants and treatment 1 smaller plants than all other treatments. Plants in the remaining treatments were very similar to each other.

Final data—average of three reps. Means were separated by the LSD test at 5% level.

| | Total fruit | | Cull fruit | Mkt. fruit | | Berry wt | |
|---|---|---|---|---|---|---|---|
| Tr. | (g/plot) | | (g/plot) | (g/plot) | | (glberry) | |
| 1 | 4174.0 | d | 460.7 | 3713.3 | d | 17.5 | b |
| 2 | 7335.3 | abc | 622.0 | 67133 | be | 19.3 | a |
| 3 | 7443.3 | abc | 456.7 | 6986.7 | abc | 19.8 | a |
| 4 | 7455.3 | abc | 492.0 | 6963.3 | abc | 20.7 | a |
| 5 | 8116.7 | ab | 593.3 | 75233 | ab | 20.8 | a |
| 6 | 7296.7 | be | 496.7 | 6800.0 | be | 20.0 | a |
| 7 | 7326.7 | abc | 573.3 | 67533 | be | 19.4 | a |
| 8 | 710.0 | bc | 442.0 | 6660.0 | bc | 20.6 | a |
| 9 | 6398.7 | c | 528.7 | 5870.0 | c | 20.5 | a |
| 10 | 8980.7 | a | 677.3 | 8303.3 | a | 20.4 | a |
| LSD_{0.05} | 1671.0 | | NS | 14921 | | 1.6 | |

There were no differences in the weight of cull berries among treatments. Culls were berries that were undersized or mis-shaped.

The average berry size did not differ as a result of the products tested. The only significant difference was in treatment 1 that received no fertilizer where berries were smaller.

Total weight of fruit (including culls and marketable fruit) and marketable weight of fruit showed nearly the same response to treatments. Pr. 10 (100% Daniels±250 EcoMin+1 SC-27) had the highest yield and Trs. 9 (75% Daniels+250 EcoMin+1 50-27) and 1(0 fertilizer control) the lowest. The remaining treatments did not differ significantly.

Daniels fertilizer alone performed the same as the conventional fertilizer at the same mineral N rate. Daniels fertilizer at 100% rate in combination with EcoMin and SC-27 gave the highest yield. Our reason for testing this combination was to determine if a biodegradable source of carbon, as exists in Daniels fertilizer, would serve as a substrate for the microbes in 80-27 or for natural soil microbes that have the facility to be enhanced by EcoMin. It appears that one or the other of these two possibilities did occur in this treatment if this is true, a source of biodegradable carbon may be the key that can consistently turn on EcoMin or 80-27. Daniels fertilizer at 75% in combination with EcoMin and SC-27 did not perform as well as the 100% Daniels rate, suggesting that nutrition was inadequate.

Neither SC-27 nor EcoMin elevated yields over the+fertilizer control levels. However, they did not lower yields either.

End of Manuscript and Experimental Description

The above manuscript demonstrates some of the inventive aspects of the present invention as set out in the following outline:

I. Summary—Page 2

A. Deeper Green Foliage

B. Compaction—Less Leaf Expansion

C. Earlier Flowering, More Flowers on Cyclamen

D. No K deficiency despite 10:3 N-K ratio vs. industry standards 10:10 N-K ratio

E. No NH₄ type N toxicity despite high (85%) ammoniacal N vs. industry standards. All mineral fertilizer @ 75% NH₄ type N exhibited expected NH toxicity problems.

II. Petunia Page 5 1.2 (Experiment #1)

A. Daniels produced the highest rated plants of all treatments.

B. Deeper green foliage with Daniels.

C. Flowering occurred earlier.

III. Bedding Plants Page 6 2.1 (Experiment #2)

A. Daniels produced a deeper green color in all (6) plants. “Deeper color has a commercial advantage”.

B. Daniels plants more compact. “Compactness is a commercial advantage

C. “The margin of safety for NFL toxicity is very large in Daniels fertilizer.”“Very resistant to NIL toxicity”.

IV. Cyclamen Page 11 3.1 (Experiment #3)

A. Significant difference in number of flowers with Daniels.

1. 9.7 Flowers v. 6.6 flowers

2. Two commercial advantages

a. send plants to market with significantly more (47%) flowers at the standard production time.

b. Send plants to market a week earlier than normal with the standard number of flowers on them.

V. Plasticulture Strawberry Trials

A. Plants fed with 100% Daniels plus ECONIM (a microbial inoculant) and SC-27 (a rock dust coated with a molasses nutrient C source). Products of Martin Marietta produced significantly higher yields of marketable berries 8,303 per plot at the same berry weight. Treatment #10.

B. Larger plants, deeper green, more leaves, foliage, when Daniels used in combination with microbial inoculant yields increased by 18.3%

The following research study demonstrates the increased crop yield (fresh wgt.) greater compactness (shorter internodes) and more “breabe” (increased mass and flowers-yield) by use of the fertilizer in an aspect of the present invention.

Pansy Growth and Mineral Nutrient Content; Daniels 10-4-3 v. Traditional Inorganic Fertility

Dr. Bill McElhannon, MMI Laboratory

Introduction: In the fall of 2000, with the assistance of 4 excellent Georgia growers, Progress Grower Supply evaluated the performance of pansies fertilizer with Daniels 10-4-3 fertilizer. Daniels is a liquid fertilizer formulated from an oil seed extract base: This fertilizer has been extensively trailed throughout the 1.15, but data reported from the trials has been primarily in a documentary form. The objective of the fall 2000 trials was to evaluate the performance of this fertilizer in a number of production environments and to have media, tissue and plant growth comparisons performed by an independent evaluator. In this case, analysis and evaluations were performed by Micro-Macro Analytical Laboratory in Athens Ga.

Pansies were grown at 4 locations. Two locations were in the metropolitan Atlanta area, one in N. Central Georgia and the other in S.E. Georgia. The 4 growers that participated in this trial were excellent pansy growers and the Daniels 10-4-3 was compared to their standard fertility programs. Nitrogen supplied from the Daniels fertilizer was equal the N supplied in the growers standard fertility program. Growers, at the various trial locations, grew different pansy varieties and had different planting schedules, but within each location, pansies used for the fertilizer comparison were planted on approximately the same date. Results and Discussion: Table I presents the fresh weights of pansies obtained from the 4 trial locations. In each trial, fertilization with the Daniels 10-4-3 produced larger plants with greater fresh weight than the pansies fertilized with the growers standard fertilizer. Increases in fresh weight varied from 11.66% with the Majestic Giants to 24.60% with the Delta Yellow variety. Consistently, all pansy varieties tested, at all locations, were more compact, with less internode elongation and more axillary breaks than that obstrvcd with the growers standard fertilizer.

TABLE 1
Fresh weights of pansies grown at 4 Georgia
production locations. Growers standard fertility program v.
Daniels 10-4-3.

| | | Weight | % increase/ |
|---|---|---|---|
| Variety | Fertilizer Fresh | grams/plant | decrease |
| Crystal Bowl Yellow | Total Grow 20-0-15 | 8.5 | +19.04% |
| Crystal Bowl Yellow | Daniels 10-4-3 | 10.5 | |
| Dart Eyed Lemon | Excel 15.2.20 | 2.6 | +16.12% |
| DarkEycd Lemon | Daniels 10-3.4 | 3.1 | |
| Delta Yellow Delta | Excel 15-2.20 | 6.1 | +24.60% |
| Yellow | Daniels 10-3-4 | 8.1 | |
| Majestic Mixed | Excel I 5-2-20 | 5.83 | |
| Majcestic Mixed | Daniels 10-3-4 | 6.60 | +11.66% |

Media and Tissue Analysis: Saturated media extract (SME) analysis of pansy media obtained from the various trial locations is reported in Table 2. The saturated media extract procedure is a method that deter-mines the soluble nutrients that are immediately available to plants. With few exceptions, nutrient levels did not vary greatly between treatments. However, the slight differences that were observed did rcsuk in consistently lower ECs (soluble salts) with the Daniels treatment This may have been due to the increased nutrient uptake by the larger plants plant grow and/or a portion of the plant nutrients were in an organic form (non ionic) that is not detected by soluble salts measurements. Growers should be advised that media EC values may be lower than that observed with conventional inorganic fertilizers when plants arc fertilized with similar rates of Daniels. Location #4 (Fable 2) is a good example, EC values with Daniels fertilization was 1.3. Fertilization with similar N rates of Excel 15-2-20 resulted in an EC of 2.1 Similar observations have been reported from other trials evaluating Daniels fertilizer and growers probably should not attempt to obtain high EC's values when fertilizing with this product. Common sense should be used here, if a grower is obtaining adequate growth with this product, do not be overly concerned if EC values arc lower than that observed with similar rates of inorganic fertilizers, In this trial, there were no consistent differences in media pH values when plants were fertilizer with the growers standard fertilizer or Daniels.

TABLE 2
Mineral nutrient analysis of pansy media obtained from 4 different growing locations.
The growers standard fertility program compared to Daniel's 10-4-3 fertilizer.

| Location | #1 | | #2 | | #3 | | #4 | |
|---|---|---|---|---|---|---|---|---|
| | Total Grow | Daniels | Excel | Daniels | Excel | Daniels | Excel | Daniels |
| | 20-0-15 | 10-4-3 | 15-2-20 | 10-4-3 | 15-2-20 | 10-4-3 | 15-2-20 | 10-4-3 |
| Nutrient | ppm | | | | | | | |
| Nitrate | 54 | 57 | 45 | 30 | 22 | 16 | 175 | 104 |
| Ammonium | 12 | 12 | 5 | 6 | 3 | 2 | 23 | 16 |
| Phosphorus | 2 | 1 | 3 | 2 | 2 | 4 | 14 | 6 |
| Potassiurn | 48 | 30 | 43 | 33 | 27 | 10 | 47 | 71 |
| Calcium | 35 | 24 | 34 | 30 | 11 | 9 | 186 | 118 |
| Magnesium | 35 | 20 | 34 | 28 | 9 | 7 | 123 | 79 |
| Iron | 0.37 | 0.16 | 0.18 | 0.17 | 0.02 | 0.15 | 0.39 | 0.16 |
| Manganese | 0.19 | 0.1 | 0.1 | 0.1 | 0.1 | 0.1 | 0.1 | 0.1 |
| Boron | 0.03 | 0.02 | 0.06 | 0.05 | 0.05 | 0.03 | 0.10 | 0.11 |
| Copper | 0.07 | 0.05 | 0.04 | 0.03 | 0.01 | 0.03 | 0.01 | 0.04 |
| Zinc | 0.59 | 0.28 | 0.29 | 0.27 | 0.11 | 0.17 | 0.03 | 0.17 |
| Molybdenum | 0.01 | 0.01 | 0.03 | 0.01 | 0.01 | 0.01 | 0.01 | 0.01 |
| Sodium | 30 | 22 | 35 | 28 | 13 | 13 | 49 | 66 |
| Aluminum | 0.01 | 0.02 | 0.24 | 0.20 | 0.06 | 0.02 | 0.35 | 0.24 |
| pH | 6.2 | 6.2 | 6.0 | 5.9 | 6.4 | 6.2 | 5.7 | 5.3 |
| EC (dS/m) | 0.8 | 0.5 | 0.7 | 0.6 | 0.3 | 0.2 | 2.1 | 1.3 |

With the exception of Iron, results from tissue analysis of pansies (Table 3) fertilized with the grower's standard fertilizer or Daniels 10-4-3 were quite similar. This is surprising because a substantial growth response was observed with the Daniels.

End of Manuscript and Experimental Description

The above manuscript demonstrates some of the inventive aspects of the present invention as set out in the following outline:

I. Summary—Page 2

A. Deeper Green Foliage

B. Compaction—Less Leaf Expansion

C. Earlier Flowering, More Flowers on Cyclamen

D. No K deficiency despite 10:3 N-K ratio vs. industry standards 10:10 N-K ratio

E. No NH₄ type N toxicity despite high (85%) ammoniacal N vs. industry standards. All mineral fertilizer @ 75% NH₄ type N exhibited expected NH₄ toxicity problems.

II. Petunia Page 5 1.2 (Experiment #1)

A. Daniels produced the highest rated plants of all treatments.

B. Deeper green foliage with Daniels.

C. Flowering occurred earlier.

III. Bedding Plants Page 6 2.1 (Experiment #2)

A. Daniels produced a deeper green color in all (6) plants. “Deeper color has a commercial advantage”.

B. Daniels plants more compact. “Compactness is a commercial advantage”.

C. “The margin of safety for NH₄ toxicity is very large in Daniels fertilizer.”“Very resistant to NH.₄ toxicity”.

IV. Cyclarnen Page 11 3.1 (Experiment #3)

A. Significant difference in number of flowers with Daniels.

1. 9.7 Flowers v. 6.6 flowers

2. Two commercial advantages

a. send plants to market with significantly more (47%) flowers at the standard production time.

b. Send plants to market a week earlier than normal with the standard number of flowers on them.

V. Plasticulture Strawberry Trials

A. Plants fed with 100% Daniels plus ECONIM (a microbial inoculant) and SC-27 (a rock dust coated with a molasses nutrient C source). Products of Martin Marietta produced significantly higher yields of marketable berries 8,303 per plot at the same berry weight. Treatment #10.

B. Larger plants, deeper green, more leaves, foliage, when Daniels used in combination with microbial inoculant yields increased by 18.3%

The following research study demonstrates the increased crop yield (fresh wgt.) greater compactness (shorter internodes) and more “breabe” (increased mass and, flowers-yield) by use of the fertilizer in an aspect of the present invention.

What is claimed is:

1. A plant fertilizer comprising:
(a) a nutrient source and biodegradable carbon source derived from the acid water produced in caustic refining of agricultural oil; and
(b) a microbial inoculant comprising nucro-orgarnsms beneficial to plant growth.

2. A plant fertilizer as recited in claim 1, wherein the microbial inoculant is carried on a substrate.

3. A method of producing plants of improved green color, comprising the steps of:
(a) cultivating plants in which a deep green color is desired; and
(b) applying a low iron fertilizer based on the acid water from caustic refining of agricultural oils.

4. A method as recited in claim 3, further comprising the step of reducing the use of pest control chemicals relative to plants grown with fertilizers not based on said acid water.

5. A method as recited in claim 3, further comprising the step of reducing the use of growth regulators relative to plants grown with fertilizers not based on said acid water.

6. A plant or animal feed produced by the process comprising the steps of:
(a) refining agricultural oil with a silicate chosen from the group consisting of sodium silicate, potassium silicate, and combinations thereof;
(b) separating the resulting soapstock from the refined agricultural oil; and
(c) using the separated product as plant feed, animal feed, or as an additive to said feeds.

7. A feed as recited in claim 6, wherein water is removed from the soapstock.

8. A feed as recited in claim 6, wherein the silicate soapstock is acidulated to make a liquid product.
